(19) United States
(12) Patent Application Publication
(10) Pub. No.: US 2021/0353473 A1
(43) Pub. Date: Nov. 18, 2021

(54) MENSTRUAL CUP

(71) Applicant: UROSILICA Co. Ltd., Daejeon (KR)

(72) Inventors: Jeong-yoon YI, Daejeon (KR); Hwa-jeong YI, Daejeon (KR); Su-ho YI, Daejeon (KR)

(21) Appl. No.: 16/874,409

(22) Filed: May 14, 2020

(51) Int. Cl. A61F 13/20

(52) U.S. Cl. CPC A61F 13/2045; A61F 13/208; A61F 2013/4562; A61F 13/2037; A61F 13/204

(57) ABSTRACT

A menstrual cup for insertion into a vaginal canal having a vaginal wall for prevention of leakage of menstrual blood may include: a body having an accommodating cavity portion opened upward is formed; a concave portion extending from the upper end of the body to gradually decrease in diameter; and a circular guide rib extending from an upper end of the concave portion to gradually increase in diameter. It can be easily inserted into the vagina so that it is deformable, and the size of the insertion direction is reduced. Not only it is effective, but when inserted it into the vagina, the circular guide rib comes into close contact with the inner wall of the vagina by restoring force for preventing a menstrual blood from leaking.

### TECHNICAL FIELD

The present invention relates to a physiological container, and more particularly, to a menstrual cup that can be inserted into the human body to receive physiological blood. In addition, the physiological container can be used for a user for suffering from urinary incontinence.

### BACKGROUND TECHNOLOGIES

In general, female's sanitary products are mainly a pad-type sanitary napkin, hygienic pad used to attach to an inner, panties, and a tampon is a feminine hygiene product designed to absorb the menstrual flow by insertion into a vagina during menstruation.

In general, a pad type sanitary napkin has advantages such as an excellent absorption and a thin thickness, since it is not firmly fixed to underwear and moves easily, there are restrictions on free activities or sports, and particularly, it is necessary to consciously restrain movements during sleep.

In addition, the pad type sanitary napkin is known to have a problem of causing dermatopathy or itching because the surface touching the skin is made of a chemical material when worn.

In addition, the insertable sanitary product has the advantage of being able to freely work in the menstrual period because it can effectively prevent the phenomenon of menstrual blood leakage compared to the pad type sanitary napkin. However, it is known that the harmful substances generated in the insertable sanitary napkin produced as a chemical component has a problem that can cause various diseases.

In addition, pad-type sanitary napkins and insert type sanitary napkins are made of a one-time product that is not reusable, so the economic burden is increasing due to the disposal cost of a large amount of waste discharge.

On the other hand, as an alternative sanitary products of the existing pad-type sanitary napkin and insert type sanitary napkin having the problems described above, an insertable menstrual cup has been developed and marketed to accommodate menstrual blood that is worn while being inserted into the vagina of a woman.

According to the related art, Republic of Korea Patent No. 10-0973524 (2010 Aug. 2), an insertable menstrual cup, which include a cup shape that the inner diameter of the wall becomes smaller from the top to the bottom, a cup-shaped body, can be inserted into a vagina of a woman during menstruation to store menstrual blood in an internal storage region, and a handle extending downward from a lower outer surface of the cup-shaped body.

However, such an insert type menstrual cup has a problem in that it is not easy to insert smoothly into a woman's vagina because the upper part of the cup-shaped body is wider than the lower part when worn.

That is, in order to insert the insert type menstrual cup into the vagina of the woman during menstruation, the upper part of the cup-shaped body is inserted into a state in which it is folded or wrinkled to be narrower than its original size. Since the upper part of the cup-shaped body is formed of a relatively hard structure to be in close contact with the inner wall of the vagina, the upper part is not easily folded or deformed. After inserting, the process of adjusting the deformed portion to be unfolded must be made indispensable, which has the inconvenience when inserting.

### PATENT DOCUMENTS

Patent Document 0001: Korean Patent No. 10-0973524 (Aug. 2, 2010)

### INVENTION DISCLOSURES

### Problems to be Solved

The present invention has been made to solve the above problems, it is possible to easily deform the guide ribs into an insertable shape using a deformation portion and/or a folding portion of a concave portion, as the folding portion is folded to be smaller in an insertion direction, it is an object of the present invention to provide a menstrual cup that can be easily inserted into the vagina by folding the guide rib and a folding portion of the concave portion.

### Means for Solving the Problems

The physiological container, according to the present invention, for achieving the above object may include: a body in which an accommodation space portion opened upward is formed therein; a concave portion extending from the upper end of the body to gradually decrease in diameter; and guide rib portion extending from the upper end of the concave portion so as to gradually expand in diameter when inserting into the vagina, the guide rib is pulled and deformed into an insertable shape, and then a front side of the guide rib is folded closely with the body so that the size of the insertion direction portion become smaller.

As shown in FIGS. 1-9, the concave portion may be formed so that a gap from the guide rib in a body direction closer to the rear side than the front side according to an aspect of the present invention.

In other word, the concave portion may be formed asymmetry from a center of the body as shown in figures.

The concave portion may include a front side height H1, a height from a guide rib to a front side concave portion and a rear side height H2, a height from the guide rib of the body direction to a rear side concave portion. The heights H1 and H2 may be formed shorter than a length L1, which is a length from a front end of the guide rib to a rear end of the guide rib.

A folding portion, may be formed at the concave portion, is formed to become thinner toward the front one side from both sides of the center portion of the front one side and the other rear according to an aspect of the present invention.

The guide rib portion may include a deformation portion formed to be thinner than the other thickness of portion of the guide rib portion to induce a shape deformation on the front one side of the upper end portion made of a ring shape according to an aspect of the present invention. The deformation portion may be formed at a rear side of the guide rib portion.

A menstrual cup may include a deformation portion, when inserting into the vagina, after pulling the deformation portion of the guide rib into a shape that can be inserted, the deformation portion of the guide rib is folded along the folding portion closely to the body so that a size of the insertion direction portion is smaller according to aspect(s) of the present invention.

The menstrual cup may have a plurality of a shape holding portion vertically and/or horizontally protruding from an inner surface and/or an outer surface of the accommodating cavity to increase the restoring force from elastic deformation according to an aspect of the present invention.

A plurality of a shape holding portion vertically and/or horizontally protruding from an inner surface and/or an outer surface of the concave portion of menstrual cup according to an aspect of the present invention.

According to an aspect of the present invention, as an example, the body may include a plurality of the shape holding portions, which are protruded from the inner surface of the accommodating cavity along the inner circumference thereof but not correspond to where a first, a second measuring units are formed.

A handle portion of menstrual cup is formed to protrude downward on the outside of the body and the handle portion may include a cavity portion, which is opened downward in the longitudinal direction therein, in the middle of the inner circumferential surface stepped to extend stepwise to both sides in the middle portion thereof according to aspect(s) of the present invention

### Effects of the Invention

According to a menstrual cup of the present invention, the guide rib portion can be easily deformed into an insertable shape through the deformable portion, during menstruation, the shape of the guide rib portion is deformable so that it can be inserted, and at the same time, the size of the portion in the insertion direction is reduced so that it can be easily inserted into the vagina of a user.

In addition, when inserted into the vagina, the guide rib portion is in close contact with the inner wall of the vagina by the restoring force, thereby preventing the menstrual blood from leaking.

In a preferred method the body is compressed during insertion. The body further has a resilience for restoring to its original shape after being inserted into the vagina.

Further, since the intermediate portion is spaced apart from the vaginal wall by a concave portion, there is an effect of minimizing friction to reduce damage of the vaginal wall and various side effects therefrom.

### BRIEF DESCRIPTIONS OF DRAWINGS

FIG. 1 is a perspective view of a menstrual cup according to an embodiment of the present invention;

FIG. 2 is a front view of the menstrual cup according to the embodiment of the present invention;

FIG. 3 is a plan view of the menstrual cup according to an embodiment of the present invention;

FIG. 4 is a side view of the menstrual cup according to embodiments of the present invention;

FIG. 5 is a side cross-sectional view of the menstrual cup according to an embodiment of the present invention;

FIG. 6 is views of menstrual cups inserted in vagina according to embodiment of the present invention;

FIG. 7 is a side cross-sectional view of the menstrual cup including plurality of shape holding portion according to an embodiment of the present invention,

FIG. 8 is a side cross-sectional view of the menstrual cup including plurality of horizontal shape holding portion and rims according to an embodiment of the present invention, and

FIG. 9 is a view showing a process of folding the menstrual cup according to an embodiment of the present invention.

### DETAILED DESCRIPTIONS

Hereinafter, with reference to the accompanying drawings, it will be described in detail with respect to the menstrual cup according to the present invention.

In adding reference numerals to the components of each drawing, it should be noted that the same reference numerals are assigned to the same components as much as possible even though they are shown in different drawings. In addition, in describing the present invention, when it is determined that the detailed description of the related well-known configuration or function may obscure the gist of the present invention, the detailed description thereof will be omitted.

In addition, in describing the components of the present invention, terms such as first, second, A, B, (a), and (b) may be used. These terms are only for distinguishing the component from other components, and the nature, order, or order of the component is not limited by the term. When a component is described as being “connected”, “coupled” or “connected” to another component, that component may be directly connected to or connected to the other component, but it should be understood that another component between each component elements may be “connected”, “coupled” or “connected.”

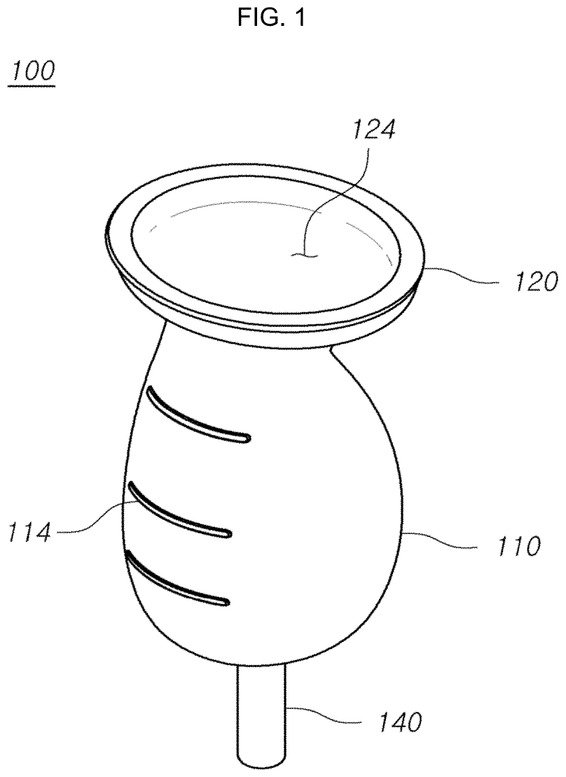

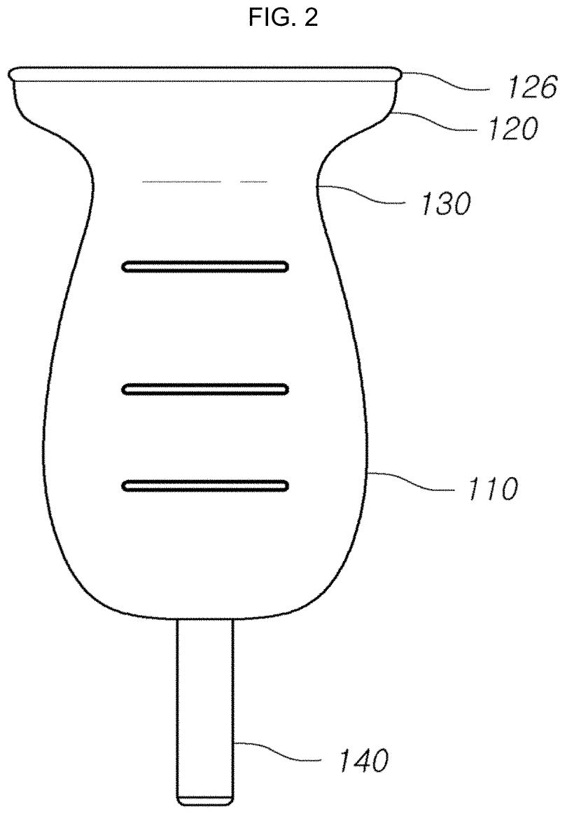

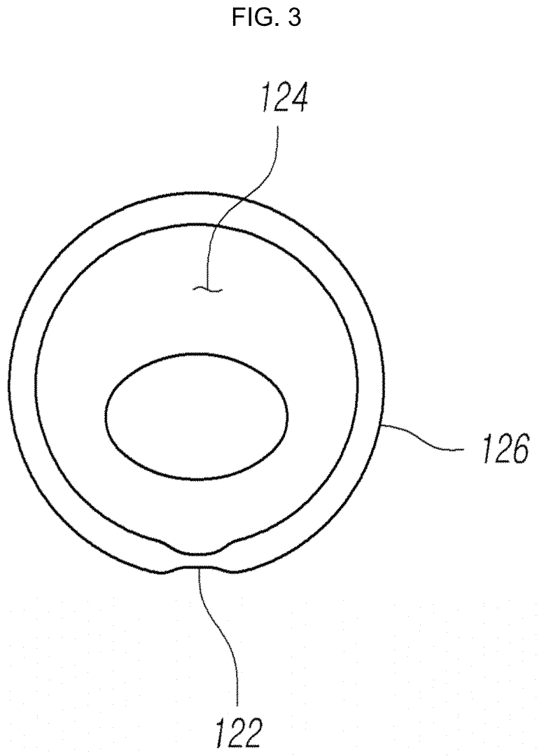

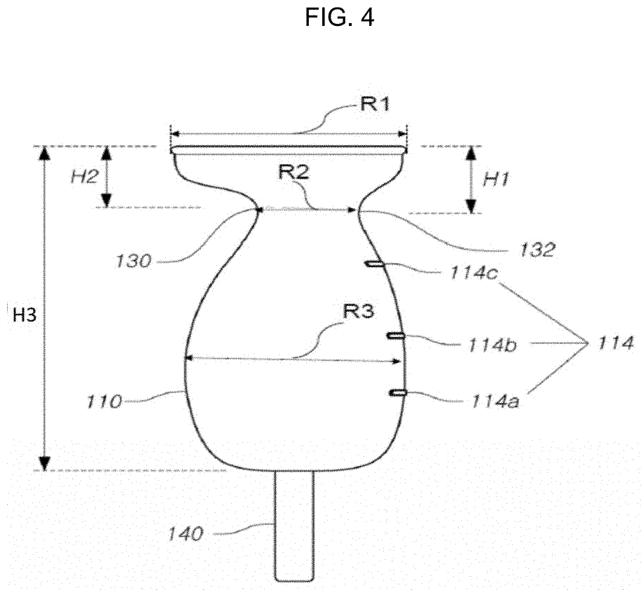

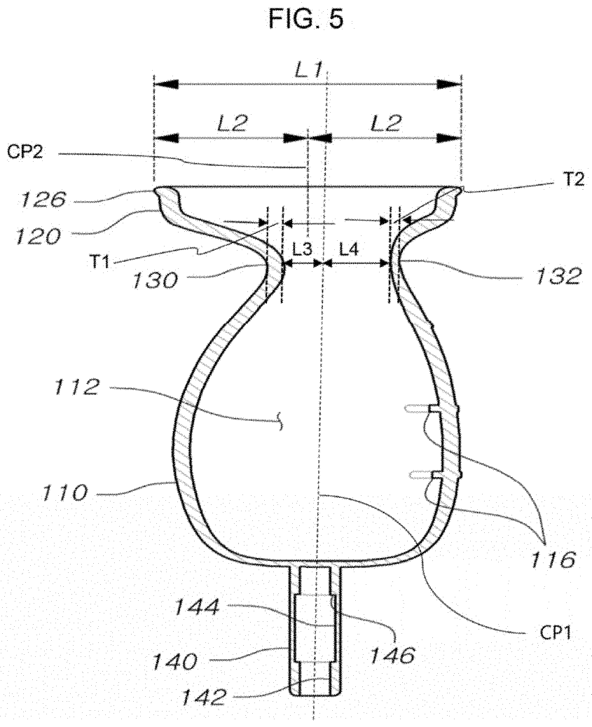

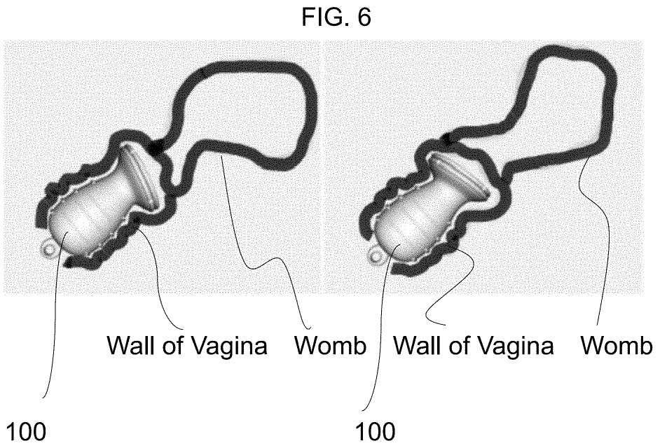

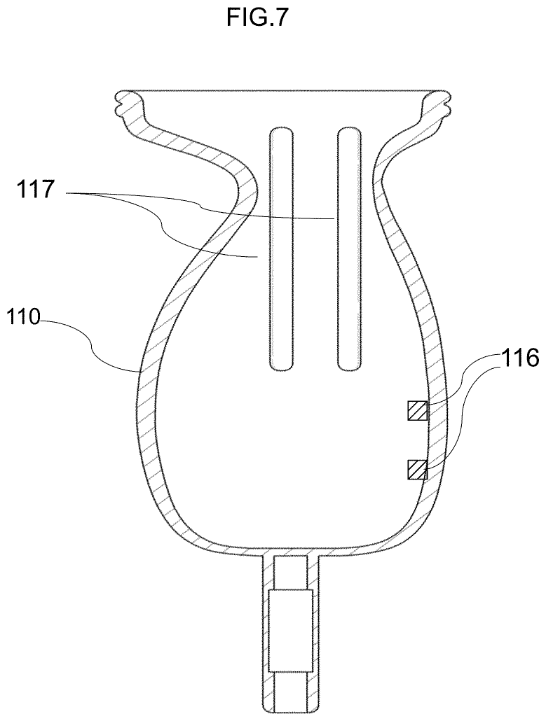

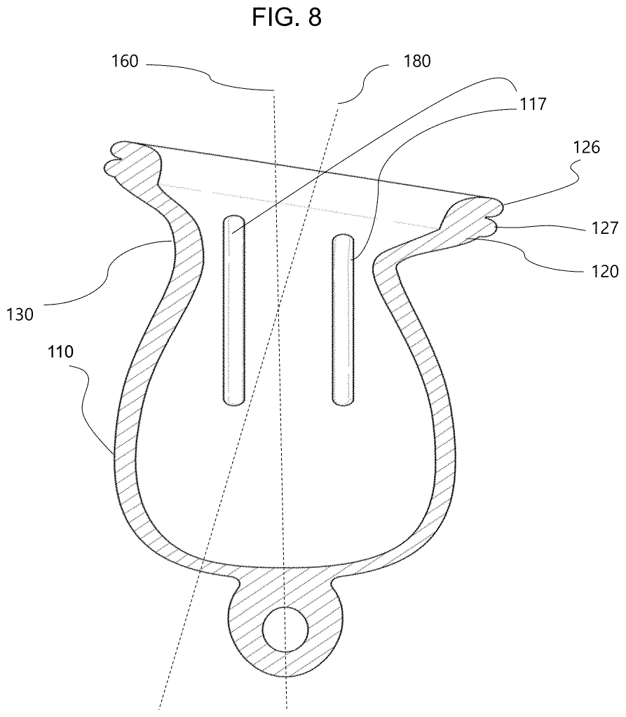

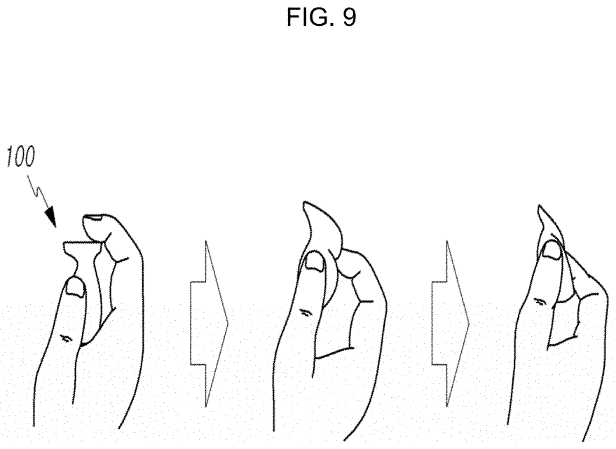

FIG. 1 is a perspective view of a menstrual cup according to an embodiment of the present invention, FIG. 2 is a front view of the menstrual cup according to an embodiment of the present invention, FIG. 3 is a plan view of the menstrual cup according to an embodiment of the present invention, FIG. 4 is a side view of the menstrual cup according to an embodiment of the present invention, FIG. 5 is a side cross-sectional view of the menstrual cup according to an embodiment of the present invention, FIG. 6 is views of menstrual cups inserted in a vagina according to embodiment of the present invention, FIG. 7 is a side cross-sectional view of the menstrual cup including plurality of shape holding portion according to an embodiment of the present invention, FIG. 8 is a side cross-sectional view of the menstrual cup including plurality of horizontal shape holding portion and rims according to an embodiment of the present invention, and FIG. 9 is a view showing a process of folding the menstrual cup according to an embodiment of the present invention. Figures are described here as only for reference, but the actual direction in use of the invention is not limited thereto.

As shown in these figures, the menstrual cup 100 according to an embodiment of the present invention may include, the body 110 is formed with an accommodating cavity 112 which is opened upwards therein; a concave portion 130 extending from the upper end of the body 110 to be gradually reduced in diameter; and a guide rib portion 120 extending from the upper end of the concave 130 so as to gradually expand in diameter.

That is, the menstrual cup 100 may include: a body 110, a concave portion 130, and a guide rib portion 120 according to aspects of the present invention.

After pulling the guide rib 120 and transforming it into an insertable shape if a user want to insert the cup 100 in a vagina of the user and then folding the front one side of the guide rib 120 closely with the body 110 so that the size of the insertion direction portion is getting smaller.

The body 110 may include an accommodating cavity 112 formed therein that is open to the upper side thereof, and the handle portion 140 protruded to the lower side therefrom.

Here, the body 110 is formed with a flat bottom surface so as not to fall easily in a state inserted into the vagina, the flat bottom is formed thinner than the side so that it is easily deformed into an oval shape when the handle 140 is pulled out.

In more detail, the entire body 110 is made of natural rubber or silicone, and an accommodating cavity 112 for storing menstrual blood therein, which is opened upwards, such as a cup shape, and a handle 140 is formed to protrude on a flat bottom outer surface according to aspect(s) of the present invention.

At this time, the body 110 is formed with one or more protrusions, a display unit 114 on one front side and/or the rear side to determine an amount of menstrual blood stored in the accommodating cavity 112.

That is, the body 110 may include at least one or more display unit 114 is formed in a vertical direction, at a front or rear portion thereof to check an amount of menstrual blood stored in the accommodating cavity 112. The display unit 114 may include a first measuring unit 114a, a second measuring unit 114b, and a third measuring unit 114c spaced in a vertical direction are formed to protrude in the left and right directions on the outer side of the front side of the body 110. An amount of menstrual blood stored in the accommodating cavity 112 can be measured by the first measurement unit 114a, the second measuring unit 114b, and the third measuring unit 114c in 5 ml, 10 ml, and 15 ml units, respectively, according to aspect(s) of the present invention.

However, it is understood that a plurality of measuring unit may be formed at the outer circumference the cup and/or inner circumference of the menstrual cup.

In addition, referring to FIG. 5, the body 110 may have a plurality of a shape holding portion 116 horizontally protruding from an inner surface of the accommodating cavity 112 to correspond to the position where the display unit 114 is formed so as to increase the restoring force during elastic deformation.

According to an aspect of the present invention, as an example, the body 110 may include a plurality of the shape holding portions 116, which are protruded from the inner surface of the body 110 along the inner circumference thereof to correspond to where a first, a second measuring units 114a, 114b are formed. However, it is understood that the shape holding portion may be formed at any location if a restoring force can be increased. For example, in addition, a plurality of shape holding portions are further vertically and/or vertically formed at the inner surface of the body 110.

The menstrual cup 110 of the present invention can be easily folded because the shape holding portion 116 is not formed on the inner surface of the accommodating cavity 112 to correspond to a position where the third measuring unit 114c is formed.

The shape holding portion 116 protrudes from the inner surface of the body 110 to correspond to the position where the first and second measuring portions 114a and 114b are formed, thus, the shape holding portion 116 can be easily returned to an original state from a deformation.

Here, the display portion 114 is formed to protrude from 0.2 mm to 0.25 mm on the other outer side of the rear of the body 110, the shape holding portion 116 may be protruded about 1 mm to 1.05 mm from an inner side of the accommodating cavity 112 at a position corresponding to the first measuring and the second measuring units 114a, 114b.

However, it is understood that the shape holding portion may be formed any other shape if it can be performed as a function of shape holding portion. The concave portion 130 is formed to gradually decrease in diameter from the upper end of the body 110. A guide rib portion 120 is formed, which is formed to be extended to gradually expand in diameter at the upper end.

That is, the concave portion 130 is formed to extend so that the diameter is gradually reduced from the upper end of the body 110, thus, the body 110 and the guide rib portion 120 in contact with the inner wall of the vagina can be spaced apart from the inner wall of the vagina to reduce a friction of the inner wall thereof.

In other words, a concave portion 130 is formed between the body 110 and the guide rib portion 120 to have a smaller diameter than both the body 110 and the guide rib 120. Thus, it is possible to reduce the contact area between the vaginal wall and the concave portion 130 due to the shape of the concave portion, i.e., by reducing the contact area with the inner wall of the vagina, the friction can be minimized to reduce damage to the inner wall and various side effects thereof.

At this time, the concave portion 130, the spaced apart from the guide rib portion 120 in the direction of the body 110 is formed closer to the rear side than the front side.

That is, the concave portion 130 has a front side gap H1, which is spaced apart from the guide rib portion 120 in a direction of the body 110 from a front side and a rear side gap H2, which is spaced apart from the guide rib portion 120 in a direction of the body 110 from a rear side. The front side gap H1 is greater than a rear side gap H2.

Here, the concave portion 130 may be formed to have the same length of gap from the guide rib 120 in a direction of the body 110 both a front side gap H1 and a rear side gap H2.

In addition, the menstrual cup 100 of the present invention, for easier folding, the front side gap H1 and the rear side gap H2 of the concave portion 130 are shorter than a length L1, from a front end of the guide rib 120 to a rear end of the guide rib portion 120.

As such, the front side and the rear side of the concave portion 130 are formed asymmetrically, if the user wants to insert into the vagina, after pulling out the guide rib and transforming it into an insertable shape, when one side of the front side of the guide rib 120 is closely folded with the body 110 so that the size of the insertion direction portion is reduced, it can be easily folded even with a small force.

In addition, a direction of the central axis of the guide rib portion and the concave portion is not in a straight line, but arranged to be optimized for the female body structure, so a feeling of the volume of the menstrual cup is much reduced when worn than the actual volume of the menstrual cup.

Further, by forming a diameter of the concave portion narrower than those of both the guide rib portion and the body, the menstrual blood can be easily collected and not spilled out.

A front and a rear portion of the shapes of the concave portion may be different from each other, so it is appropriately designed according to a position and a body structure of the cervix.

The shortest diameter R2 of the concave portion 130 may be greater than ⅓ of the maximum diameter R3 of the body 100 and the concave portion is located at a distance H1, H2, which is shorter than a radius of the longest distance of the concave or a radius of the longest of the guide rib portion R1.

Thus, after the guide rib portion is folded up and down, and then the concave portion can be wrapped with the body 110. Thus, the menstrual cup can be double-folded according to an aspect of the present invention.

The reason that the minimum diameter R2 of the concave portion should be larger than ⅓ of the maximum diameter R3 of the body is that it is difficult to make and remove the menstrual cup from the mold if the body is more than 3 times larger than the concave portion.

The reason that the position of the concave portion 130 should be at the position of H shorter than ½ length of R1, i.e., a radius of the guide rib portion and ½ length of H3, from the top guide rib portion to a bottom of the body. Thus, the guide rib portion can be folded up and down and wrapped by the left and the right of the body. If it is in a place larger than ½ length of R3, it is difficult to fold it and wrap it from side to side with the body 110.

In addition, as shown in FIG. 5, the concave portion 130 may include a folding portion 132 formed to be gradually thinner from both sides of the center portion of the front one side and the other rear to the front side.

A thickness of a folding portion 132 T2 is thinner than a thickness T1 of the other portion of the concave portion 130 to be folded easily according to aspect(s) of the present invention.

In addition, as shown in FIGS. 7 and 8, a plurality of vertical bar shape holding portions 117 may be formed on an inner circumference of the concave portion with predetermined horizontal intervals.

The plurality of vertical bar shape holding portions 117 may be formed above where the horizontal shape holding portions 116 are formed as shown in FIG. 7.

Lengths of the plurality of vertical bar shape holding portions may be predetermined lengths according to size and/or shape of the body.

The Lengths of the plurality of vertical bar shape holding portions may be different from each other based on shape of the body and concave portion.

The folding portion 132 is a portion of the concave portion 130 that is formed to become thinner as the thickness increases toward the front side from both sides of the middle portion of the front side and the rear side and deformed into an insertable shape when the guide rib 120 is pulled.

That is the folding portion 132 is a part formed with a thickness thinner than the thickness of the other side of the rear portion so that it can be folded more easily by reducing the restoring force to return to an original state.

When the front side of the guide rib 120 is folded close to the body 110 so that the size of the insertion direction portion is small, it reduces the restoring force to return to the original state and has a thickness thinner than the thickness of the other side of the rear side for easier folding.

At this time, the concave portion 130 may be formed between a guide rib portion 120 and about an upper half of the body 110. In other word, the concave portion 130 may be formed above half portion of a length, H3, from a top of the guide rib portion 120 to the bottom portion of the body 110 as shown in FIG. 4.

In addition, the folding portion 132 is preferably formed in a range of a length L2 from one front end to an upper half of a length L1 from the front end to the rear end of the guide rib portion 120 as shown in FIG. 5.

Thus, as clearly shown in FIG. 5, a center point line of the guide rib portion CP2 is not the same as a center point line of an x-axis of the body CP1. In addition, a center point line of the concave portion is also deviated from the center point line of the x-axis of the body CP1 because the lengths of the L3 and L4 are not the same. In other words, the menstrual cup is formed asymmetrically to prevent the menstrual blood from leaking efficiently based upon a shape of a vagina.

Here, the concave portion 130 may be formed to become thinner from both sides of the center portion of the front side and the rear side toward the rear side.

The thickness of the front side and the rear portion of the other side from the front side and the rear side may be formed to be gradually decreased.

That is, the folding portion 132 may be formed at a front side, a rear side, or both the front and rear sides of the concave portion 130.

However, it is understood that the folding portion may be formed at the concave portion 130.

Subsequently, the guide rib portion 120 extends from the upper end of the concave portion 130 so as to gradually expand in diameter, and is formed in a shape in which the thickness of the upper end of the guide rib portion is increased in a ring shape so that a restoring force is increased during elastic deformation.

That is, the guide rib portion 120 is formed to extend gradually in diameter from the upper end of the concave portion 130, the upper end of the guide rib portion is formed with a rim 126 protruding in a ring shape along upper portion the guide rib portion 120 so that the restoring force is increased when elastic deformation.

At this time, an outer diameter of the guide rib portion 120 is formed larger than an outer diameter of the body 110, when inserted into the vagina of a user, close to the inner wall of the vagina to prevent the leakage of menstrual blood to the outside of the user.

Accordingly, the guide rib portion can be in close contact with the inner wall of the vagina and prevent the menstrual cup 100 inserted into the vagina from being pulled out from the vagina accidently, thereby preventing the menstrual blood from leaking out.

Here, when the thickness of the guide rib portion 120 is formed thin to fold the guide rib portion 120 well, the restoring force is lowered and the airtightness between the vaginal inner wall and the guide rib 120 is reduced. Thus, the guide rib portion 120 is to increase the thickness of the upper end in a ring shape rim 126 so that the restoring force is increased during elastic deformation, it is possible to improve not only a restoring force but also a fixing force between the vaginal inner wall of the user according to an aspect of the present invention.

At this time, the guide rib portion 120 may include a deformation portion 122, which is formed to be thinner than the other thickness of the rear side to induce a shape deformation on the front upper end portion, which is in a ring shape.

The deformation portion 122 is formed at a front end of the guide rib 120 and a thickness of the deformation portion is thinner than a thickness of the rear end of the guide rib portion 120, and is made of a ring shape so that the guide rib portion 120 can be easily transformed into an insertable shape.

The deformation portion 122 is formed at a front upper end of the guide rib portion 120 and to be thinner than a thickness of a rear upper end of the guide rib portion 120.

As described above, the menstrual cup 100 may increase a strength thereof by thickening the thickness of the upper end of the guide rib portion 120, and can easily deform the shape by reducing the thickness of a front upper end portion of the guide rib portion 120, it can be easily folded and inserted into the vagina, and after inserted into the vagina, the guide rib portion 120 is returned back to the original shape by the restoring force, thus, it can prevent from leaking menstrual blood as the guide rib portion closely contacted to the inner wall of the vagina according to an aspect of the present invention.

That is, the menstrual cup 100 can easily deform the guide rib portion 120 into an insertable shape through the deformation portion 122, and can be folded to reduce the size of the insertion direction portion through the folding portion 132, when inserted into the vagina, the guide rib 120 portion is in close contact with the vaginal wall by the restoring force, thereby preventing the menstrual blood from leaking.

A plurality of deformation portion 122 may be formed according to another aspect of the present invention.

The handle portion 140 is protruded downwardly on an outside of the body 110, a cavity portion is formed in a lower side in a longitudinal direction therein, the grooves 144 is formed extending stepwise in both longitudinal directions in a middle of an inner circumferential surface 142.

More specifically, the handle portion 140 has a pipe shape, one end of which is connected to the outer lower side of the body 110, and has a space portion formed therein, which is opened downward along the longitudinal direction, and a groove 144 extending stepwise is formed in both longitudinal directions in a middle of an inner circumferential surface 142.

Accordingly, the menstrual cup 100 of the present invention, when holding the handle portion 140, by the groove 144 formed therein, a thickness of the middle side thereof is more reduced than a thickness of both sides in a longitudinal direction, so that it can be gripped more firmly when pulling the handle portion 140. Thus, it is possible to pull without slipping by a step 146 formed on both sides of the groove 144.

In addition, a plurality of rims 126, 127 may be formed vertically on the guide rib portion 120 as shown in FIG. 8. As an example, a first rim 126 and a second rim 127 are formed vertically spaced apart to be unfolded easily after the menstrual cup 100 inserted into the vagina as shown in FIG. 8. In addition, the menstrual cup 100 may prevent the menstrual blood from leaking by forming a plurality of rims according to an aspect of the present invention.

The plurality of the rims 126, 127 may be formed on the guide rib portion so that the menstrual cup can be made smaller. Thus, the vagina may be less contacted and less irritated by forming the first and the second rims 126, 127 closely each other.

In addition, the plurality of the rims may easily unfolded after the menstrual cup 100 into the vagina by the restoring force.

Such the guide rib portion 120 of a menstrual cup 100 can easily be deformed into an insertable shape through the deformation portion 122 and can be folded to reduce the size of the insertion direction portion through the folding portion 132. As a result, during menstruation, the shape of the guide rib 120 is deformable to be insertable, and at the same time, it can be easily inserted into the vagina so that the size of the insertion direction portion becomes smaller.

In addition, when inserted into the vagina, the guide rib portion 120 is in close contact with the inner wall of the vagina by the restoring force, thereby preventing the menstrual blood from leaking.

In addition, the concave portion may be formed asymmetry from a center of the body as shown in FIG. 8.

Further, by the concave portion 130, the middle portion of the menstrual cup is separated from the inner wall of the vagina, thereby minimizing friction, it is possible to reduce damage to the inner wall of the vagina and various side effects.

The menstrual cup 100 is integrally formed with the body 110, the concave portion 130 and the guide rib portion 120 formed of the same material to store the menstrual blood in the accommodating cavity 112 through the opening 124. Unlike this, the body 110, the concave portion 130, and the guide rib portion 120 may be formed in a single structure with different materials according to aspect(s) of the present invention.

The usage of aspect(s) of the present invention having the above configuration will be described with reference to FIGS. 3-9.

First, when the menstrual cup 100 is to be inserted into the vagina, as shown in FIG. 9, the deformation portion 122 of the guide rib portion 120 is pulled and deformed into an insertable shape. The deformation portion 122 of the guide rib portion 120 is folded along the folding portion 132 in close contact with the body 110 to be smaller.

Then, the menstrual cup 100 is inserted into the vagina so that the guide rib portion 120 is in close contact with the inner wall of the vagina by the restoring force.

And, if the user wants to remove the sanitary container 100, grab the handle 140 of the menstrual cup 100 and pull the same for removing the menstrual cup 100.

Without having the deformation portion 122 of the guide rib portion 120, folding any portion of the guide rib portion 120 and folding toward the folding portion 132 again the menstrual cup 100 to be inserted.

Further, as shown FIG. 8, the guide rib portion, the concave portion, and the body are not aligned each other. In other words, the centers of x-axis of the guide rib portion 160 and the body 180 were not placed on the same straight line.

Thus, after the menstrual cup was inserted, the directions of the pushing force on the menstrual cup by the wall of the vagina are distributed.

Accordingly, the menstrual cup does not come out by turning the directions of the pushing force by the wall of the vagina on the menstrual cup, thereby the menstrual cup is maintained in the vagina with safe.

In the above, even if all the components constituting the embodiment of the present invention are described as being combined or operated as one, the present invention is not necessarily limited to these embodiments. That is, within the object scope of the present invention, all the components may be selectively combined to operate.

In addition, the terms “include”, “consist” or “have” as described above mean that the corresponding component can be inherent, unless specifically stated otherwise, not to exclude other components. It should be interpreted as being able to further include other components. All terms, including technical or scientific terms, unless otherwise defined, have the same meaning as commonly understood by a person skilled in the art to which the present invention pertains. Commonly used terms, such as predefined terms, should be interpreted as being consistent with the meaning in the context of the related art, and are not to be interpreted as ideal or excessively formal meanings unless explicitly defined in the present invention.

The above description is merely illustrative of the technical idea according to aspect(s) of the present invention, and those skilled in the art to which the present invention pertains may make various modifications and variations without departing from the essential characteristics of the present invention. Therefore, the embodiments disclosed in the present invention are not intended to limit the technical spirit of the present invention, but to explain, and the scope of the technical spirit of the present invention is not limited by these embodiments. The scope of protection of the present invention should be interpreted by the claims below, and all technical spirits within the scope equivalent thereto should be interpreted as being included in the scope of the present invention.

### EXPLANATION OF THE REFERENCE NUMERALS

100: menstrual cup
110: body
112: accommodating cavity
114: display unit
116: a horizontal shape holding portion
117: a vertical shape holding portion
120: guide rib portion
122: deformation portion
124: opening
130: concave portion
132: folding portion
140: handle portion

What is claimed is:

1. A menstrual cup for insertion into a vaginal canal having a vaginal wall for prevention of leakage of menstrual blood, comprising:
a body having an accommodating cavity portion opened upward is formed;
a concave portion extending from the upper end of the body to gradually decrease in diameter; and
a circular guide rib portion extending from an upper end of the concave portion to gradually increase in diameter.

2. The menstrual cup of claim 1, further comprising:
a folding portion formed at the concave portion, wherein the folding portion is relatively thinner than the other portion of the concave portion.

3. The menstrual cup of claim 1, wherein the circular guide rib portion has a deformation portion formed at the circular guide rib, wherein the deformation portion has a thickness relatively thinner than other portion of the thickness of the guide rib portion so that may be easily transformed into an insertable shape.

4. The menstrual cup of claim 1, wherein the concave portion is asymmetrically formed.

5. The menstrual cup of claim 4, wherein a front portion of the concave portion is relatively shorter than a rear portion of the concave portion such that a height of front portion of the circular guide rib portion and a height of the rear portion of the circular rib portion are different from each other.

6. The menstrual cup of claim 4, wherein a front side and a rear side portions of the concave portion are asymmetrically formed with respect to a center line of an x-axis of the body.

7. The menstrual cup of claim 1, wherein the concave is formed at a distance from a top of the guide rib portion shorter than a radius of the guide rib portion and ½ times of a length from the top guide rib portion to a bottom of the body, and a minimum diameter of the concave portion is longer than one third of the maximum diameter of the body.

8. The menstrual cup of claim 1, wherein a front side and a rear side portions of the concave portion are asymmetrically formed with respect to a center line of an x-axis of the body.

9. The menstrual cup of claim 1, further comprising:
the plurality of shape holding portion is vertically formed with predetermined horizontal intervals on the body.

10. The menstrual cup of claim 9, further comprising:
a plurality of shape holding portion is horizontally formed with predetermined vertical intervals on the body.

11. The menstrual cup of claim 4, further comprising:
a plurality of shape holding portion is horizontally formed with predetermined vertical intervals on the concave portion.

12. The menstrual cup of claim 4, further comprising:
a plurality of shape holding portion is formed inner circumferential surface of the concave portion and sizes of the plurality of shape holding portions are different from each other.

13. The menstrual cup of claim 1, further comprising:
a plurality of shape holding portion is horizontally formed with predetermined vertical intervals on the concave portion and some of the plurality of shape holding portion is vertically formed with predetermined horizontal intervals on the body.

14. The menstrual cup of claim 1, further comprising a plurality of a shape holding portion horizontally protruding from an inner surface of the accommodating cavity.

15. The menstrual cup of claim 1, further comprising a first rim is formed at an end of the circular guide rib portion.

16. The menstrual cup of claim 15, further comprising a second rim is formed next to the first rim.

17. The menstrual cup of claim 1, further comprising:
a deformation portion is formed on the guide rib portion.

18. The menstrual cup of claim 1, wherein the guide rib portion is asymmetrically formed and wherein an x-axis of the guide rib portion and an x-axis of the body are not aligned each other.

19. The menstrual cup of claim 1, further comprising:
a plurality of measurers formed on the menstrual cup.

20. The menstrual cup of claim 1, further comprising:
a handle formed to protrude downward on the outside of the body, wherein the handle includes a cavity portion, which is opened downward in a longitudinal direction therein, in a middle of an inner circumferential surface of the handle stepped to extend stepwise to both sides in the middle portion thereof.
